(19) United States
(12) Patent Application Publication
(10) Pub. No.: US 2025/0237584 A1
(43) Pub. Date: Jul. 24, 2025

(54) IMPROVED PROCESS FOR PROCESSING BIOLOGICAL SAMPLES

(71) Applicant: DIAPATH S.P.A., Martinengo (BG) (IT)

(72) Inventors: Carmelo Lupo, Martinengo (BG) (IT); Nadia Casatta, Martinengo (Bg) (IT); Giorgio Manenti, Martinengo (BG) (IT); Paolo Danzi, Martinengo (Bg) (IT); Alberto Battistel, Martinengo (BG) (IT); Vladimiro Bergamini, Martinengo (BG) (IT)

(73) Assignee: DIAPATH S.P.A., Martinengo (BG) (IT)

(21) Appl. No.: 18/703,377

(22) PCT Filed: Oct. 25, 2022
(86) PCT No.: PCT/IB2022/060245

§ 371 Date: Apr. 22, 2024

(30) Foreign Application Priority Data
Oct. 29, 2021 (IT) 102021000027884

(51) Int. Cl. G01N 1/36; G01N 1/30

(52) U.S. Cl. CPC G01N 1/36; G01N 1/30; G01N 2001/364

(57) ABSTRACT

Subject-matter of the present invention is an improved process for processing a biological sample, in particular a process comprising the rotation around its own axis of said biological sample.

### SUMMARY OF THE INVENTION

Subject-matter of the present invention is an improved process for processing a biological sample, in particular a process comprising the rotation around its own axis of said biological sample.

### BACKGROUND ART

The anatomo-pathological diagnosis is the result of the interpretation by the anatomo-pathologist of the morphological, macroscopic and microscopic characteristics of the biological sample under examination.

To provide an accurate and complete diagnosis, the sample excised from the patient must undergo a series of treatments with the purpose of ensuring its preservation over time. Such treatments essentially provide for:
i. fixation;
ii. dehydration;
iii. clarification; and
iv. paraffin infiltration.

The samples, inside appropriate cassettes, are immersed for their processing for a certain period of time in the reagent suitable for each treatment, then they are collected and immersed in the reagent for the next treatment.

With the intent of improving the processing of biological samples, various reagents and technologies have been developed.

As far as reagents are concerned, the Applicant has recently filed two Italian Patent Applications (IT102020000025159 and IT102020000031031, extended as Patent Application WO2022/084907) directed to compositions that allow to make simultaneous treatments of dehydration, clarification and paraffin infiltration of biological samples, resulting in more homogeneously processed biological samples while saving time and solvents.

On the other hand, relative to the technologies, devices have been proposed that process the biological sample by immersion in the appropriate reagent not statically, as in the conventional processing, but dynamically.

U.S. Pat. No. 6,902,928 describes an instrument for processing histological samples constructed so that the samples are arranged along the walls of a cylindrical (or truncated-cone) chamber that can be opened on one of the two bases in such a way that the samples can be extracted even in the presence of the solution, with which they are treated, that remains at the bottom of said chamber. The samples are immersed in the solution thanks to the rotation of the chamber itself, which allows all samples to pass sequentially through the area in which the solution is present. However, in this device, the samples are not continuously immersed in the solution, and this can result in drying the sample itself with consequent damage. Furthermore, processing different samples in the same reagent solution is cause of contamination and can lead to incorrect analysis.

WO2020250121 describes a device for promoting the passage of a reagent solution through a biological sample thanks to the fact that said sample is moved inside the solution itself by the use of a specific holder in which it is housed and subjected to a motion of revolution. This way, the samples, housed in the described holder, are exposed to a convective motion of the solution that allows the latter to penetrate inside the sample itself. Even this solution has drawbacks, such as the need to use large amounts of reagent solutions and, as in the case of U.S. Pat. No. 6,902,928, the high risk of contamination.

US2010/009349 describes a method for processing a biological sample that comprises the use of a polyol and at least one additive as reagents. Much of the description is addressed to the type of reagent used for processing. Processing apparatuses are also reported, in which there are rotating media (“mixing devices”) to mix reagents around a sample that is enclosed in a cassette (“immersion aid”) inside a processing container. This document reports that the rotation occurs at 10 rpm (paragraph [0076]), that is, at an extremely low speed.

US2014/0193314 describes an automated apparatus for extracting a sample from the cassette after the infiltration step, which comprises heating the cassette and exposing it to mechanical vibrations to facilitate the sample removal.

US2011/0250634 describes a container for storing samples that are to be processed at a later time, which substantially comprises two housings, one for the sample and one for the reagents. Such housings are separated by a membrane that can be broken through a rotary motion of means inside the container, thus allowing reagents to come into contact with the sample to perform processing.

None of the above documents allows for rapid and efficient processing of biological samples.

Therefore, there is still a need to develop new techniques for processing biological samples that overcome the drawbacks of the prior art, thus providing samples processed homogeneously in a short time, using minimal amounts of reagents and eliminating the risks of damage and contamination of the reagents.

### OBJECTS OF THE INVENTION

An object of the present invention is to provide a process for processing biological samples that allows the complete and homogeneous fixation and/or dehydration and/or clarification and/or paraffin infiltration of said samples quickly, by using minimal amounts of reagents and eliminating the risk of damage.

A further object of the present invention is to provide a process for processing biological samples that allows the complete and homogeneous fixation and/or dehydration and/or clarification and/or paraffin infiltration of said samples while eliminating the risks of contamination among said samples.

### BRIEF DESCRIPTION OF THE FIGURES

FIG. 1 shows a side view of an example of a closed device useful in the process of the invention.

FIG. 2 shows a top view of the device of FIG. 1, without the lid.

FIG. 3 schematically shows the difference between the motion to which the sample is subjected according to the method of the invention (a) and the motion to which the sample is subjected according to the prior art described in WO2020250121 (b) (top view); the arrows show the direction of the liquid flow determined by the motion of the sample itself.

FIG. 4 shows three of the possible configurations of the axes around which the sample rotates.

### DESCRIPTION OF THE INVENTION

According to one of its aspects, subject-matter of the invention is a process for processing a biological sample comprising the rotation around its own axis of said biological sample immersed in a processing reagent.

According to the present invention:
by “processing” is meant to denote the treatments of fixation and/or dehydration and/or clarification and/or paraffin infiltration, whether done singly, sequentially or simultaneously;
by “biological sample” is meant to denote any biological, cytological, histological, and autopsical sample to be subjected to an anatomo-pathological analysis;
by “rotation around its own axis” is meant to denote that the biological sample is subjected to a circular motion around its own axis;
by “processing reagent” is meant to denote a compound or mixture of compounds in the liquid state, which are suitable for processing biological samples as is defined herein.
by “axis” is meant to denote a straight line passing through the center of mass of the sample in any direction, for example in longitudinal, transverse, diagonal, sagittal direction, etc.

According to the invention, in the process of the invention, the biological sample is placed inside a container for biological samples.

According to the invention, the container for biological samples containing the biological sample is placed inside vessels containing the processing reagent, which in turn are housed in a processing device equipped with means for moving the cassette around its own axis.

According to the invention, the container for biological samples is rotated around its own axis inside an appropriate vessel, for example but not limited to, a cylindrical vessel, containing the processing reagent, by any possible means suitable for the purpose.

According to a preferred embodiment shown in the attached Figures, the container for biological samples containing the sample is rotated around its own axis by rotating means connected to the processing device.

According to a preferred embodiment, each biological sample is processed separately from the others.

A preferred container for biological samples according to the invention is a cassette for biological samples.

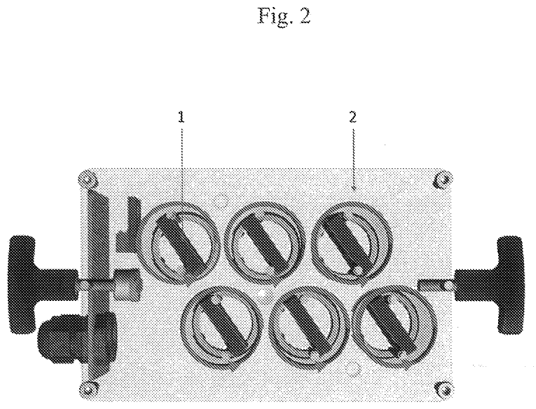

With particular reference to the embodiment of the Figures, a cassette (1) containing the biological sample is inserted into a cylindrical vessel (2) that contains the reagent and is moved thanks to one or more rotating rods (3); the top view shown in FIG. 2 allows to visualize the rotating movement of the cassettes inside said vessel. This way, the flow of reagent hits the sample as it rotates around its own axis. The rods (3) rotate, causing in turn the cassette containing the biological sample to rotate, keeping its center of mass stationary.

Furthermore, the rotation of the container for biological samples results in continuous agitation of the reagent; therefore, even in the case a mixture of reagents is used, the components of said mixture would remain homogeneously dispersed among themselves thus allowing proper sample processing.

As shown in the Figures, by way of illustration only, multiple samples can be processed simultaneously inside a fit-for-purpose device, but each sample is preferably processed individually in a single vessel, to avoid contamination risks. This technical solution is an important advantage over the devices of the prior art.

As mentioned, the mechanism described herein with reference to the attached Figures is by way of example only, and any other system that allows the rotation of the sample around its own axis inside a processing reagent is included in the inventive concept and is within the scope of protection of the present invention.

According to an embodiment, the rotation of the sample can be carried out in any way or direction, depending on how the rotation axis is positioned inside the processing reagent. By way of example, the sample can be rotated clockwise or counterclockwise, and the rotational direction can be alternated during processing. Therefore, still by way of example, after a first rotation for an appropriate period of time in one way, for example clockwise, the rotation of the sample can be stopped and resumed in the opposite way, for example counterclockwise, for another appropriate period of time. This embodiment can allow even more homogeneous processing of the sample.

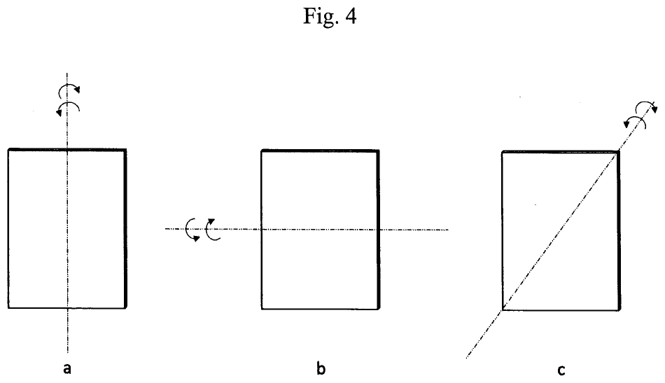

Furthermore, the rotation axis can also be varied during processing, keeping the transition from the center of mass fixed. In FIG. 4 three of the possible configurations of the axis around which the sample rotates, are exemplified.

The process of the invention, which involves rotating the sample along its own axis, allows to subject said sample to higher rotation velocities than the velocities allowed by the methods of the prior art. In fact, it was surprisingly found that with the process of the invention it is possible to significantly increase the speed of rotation of the sample while causing no damage or alteration to it.

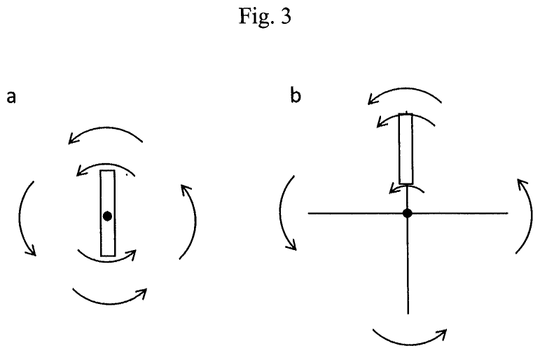

In fact, in the process of the invention, the rotational speed can range from 50 to 5,000 rpm (rotations per minute), preferably from 100 to 1,000 rpm, for example, from 200 rpm up to even 300, 400 or 500 rpm, in contrast to the process of the prior art (WO2020/250121), which uses a motion of revolution of the sample and not a rotational motion around its own axis (thus keeping the center of mass stationary), the speed is only 150 rpm. A diagram of the difference between the rotational motion of the present invention and the motion of revolution of the prior art is provided in FIG. 3 (shown as a) and b), respectively).

The rotational speed of the sample of the process of the present invention is significantly higher even than that used in US2010/009349, which is only 10 rpm. Such a speed has no influence on the sample processing, in contrast to the speed used herein, which provides excellent and rapid processing. This aspect is especially true in complex samples wherein the porosity, constrictiveness and tortuosity of the internal structures of the sample itself prevent linear fluid exchange. In fact, where the structure and composition prevent good porosity of the biological tissue, the permeability to the different components and the consequent replacement of water in the tissue with paraffin can only be ensured by a more thorough rotation, thus aiding the processing of the different components quickly and with excellent quality.

According to a preferred embodiment, within the above range, the rotational speed of the sample is modulated, preferably increased, during processing. By way of example, it is possible to initiate sample processing by setting a rotational speed of 250 rpm and by increasing it to 450 rpm during processing; by way of example, the speed can be increased by 50 rpm every 20 minutes or so.

As mentioned, the processing process of the invention comprises the treatments of fixation and/or dehydration and/or clarification and/or paraffin infiltration. Such treatments can be carried out individually or sequentially.

The treatments of dehydration and/or clarification and/or paraffin infiltration can also be carried out simultaneously.

According to an embodiment, the treatments of dehydration and/or clarification and/or paraffin infiltration are carried out simultaneously by using a single processing reagent. According to a more preferred embodiment, said single processing reagent is that described in IT102020000025159 comprising paraffin, at least one alcohol selected from ethyl alcohol and isopropyl alcohol and at least one hydrocarbon selected from naphtha and octane or in IT102020000031031 comprising paraffin, at least one alcohol selected from ethyl alcohol and isopropyl alcohol and at least one hydrocarbon selected from isoparaffin and limonene.

According to a preferred embodiment, said single processing reagent comprises, or alternatively consists of:
paraffin (as defined above): 40-60%, preferably about 50%;
ethyl alcohol+isopropyl alcohol: 15-25%, preferably about 20%;
at least one hydrocarbon (as defined above): 25-40%, preferably about 30%, said hydrocarbon being selected from naphtha, octane, isoparaffin and limonene; and
optionally one or more further components, at the rate of 0-15%, preferably 0-5%;
said % being expressed in volume with respect to the total volume of the composition.

According to another preferred embodiment, said single processing reagent comprises, or alternatively consists of:
paraffin (as defined above): 40-60%, preferably about 50%;
ethyl alcohol: 15-25%, preferably about 20%;
at least one hydrocarbon (as defined above): 25-40%, preferably about 30%, said hydrocarbon being selected from naphtha, octane, isoparaffin and limonene; and
optionally one or more further components, at the rate of 0-15%, preferably 0-5%;
said % being expressed in volume with respect to the total volume of the composition.

According to another preferred embodiment, said single processing reagent comprises, or alternatively consists of:
paraffin (as defined above): about 50%;
ethyl alcohol: about 20%;
isoparaffin: about 30%; and
optionally one or more further components, at the rate of 0-15%, preferably 0-5%;
said % being expressed in volume with respect to the total volume of the composition.

According to a preferred embodiment, in said single processing reagent, said at least one hydrocarbon comprises isoparaffin.

According to a preferred embodiment, said single processing reagent does not comprise any ketone, for example does not comprise acetone.

According to a preferred embodiment, said single processing reagent comprises, or alternatively consists of 50% paraffin, 5% ethyl alcohol, 15% isopropyl alcohol and 30% isoparaffin.

According to a preferred embodiment, said single processing reagent comprises, or alternatively consists of 50% paraffin, 20% ethyl alcohol and 30% isoparaffin.

According to a preferred embodiment, said single processing reagent comprises, or alternatively consists of 43-50%, preferably about 45-50% paraffin, 15% ethyl alcohol, 5% isopropyl alcohol, 30% at least one hydrocarbon as defined above, preferably comprising or consisting of isoparaffin and optionally one or more further components at the rate of 0-15%, preferably 0-5%.

As explained in the patent applications cited above, said single processing reagent allows to simultaneously perform the treatments of dehydration, clarification and paraffin infiltration, therefore incorporating in a single process the treatments (ii), (iii) and (iv) which conventionally involve several passages in solvents and in paraffin. In practice, after an optional first washing of the sample previously fixed with formalin, with hydroalcoholic solution (50% or 70%), the single processing reagent dehydrates, clarifies and infiltrates the sample in a single step followed by an optional drying step before the inclusion in paraffin block.

The processing times are of course variable depending on the size of the biological sample. By way of example, samples about 3 mm thick can be processed with the single processing reagent mentioned above in a time of about 90 minutes.

The invention will be now described in detail in the Experimental Section below, by way of illustration only and in no way limiting.

### Experimental Section

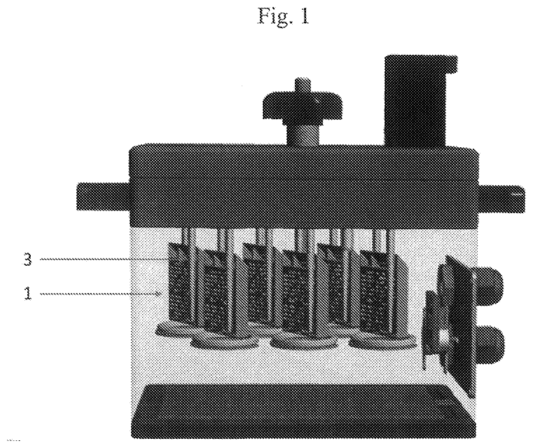

The following are examples on how to process samples of different kinds. Samples 3 mm thick were fixed in 4% formalin and processed by immersion in 40 ml of a solution constituted by 50% paraffin, 20% ethyl alcohol and 30% isoparaffin in an apparatus such as that shown in FIGS. 1 and 2. The samples were subjected to alternating clockwise and counterclockwise rotation, with an initial speed of 250 rpm, increasing by 50 rpm every 20 minutes, up to the maximum speed of 450 rpm, for 90 minutes in total. The procedure allowed to accomplish excellent sample processing, comparable to that of time-consuming and complex traditional processing.

Alternatively, biopsy samples 1 mm thick were fixed in 4% formalin and processed by immersion in 40 ml of a solution constituted by 50% paraffin, 20% ethyl alcohol and 30% isoparaffin in an apparatus such as that shown in FIGS. 1 and 2. The samples were subjected to alternating clockwise and counterclockwise rotation, with an initial speed of 250 rpm, increasing by 50 rpm every 5 minutes, up to the maximum speed of 400 rpm, for 15 minutes in total. The procedure allowed to accomplish excellent sample processing, comparable to that of time-consuming and complex traditional processing.

During the experiments and from the results obtained from literature searches, it was found that depending on the density and composition of the tissue, the sample can be processed in more or less time. A sample 3 mm thick of lung tissue for example, being a hollow organ with a structure that in itself facilitates the flow of reagents, can be processed with a timeline significantly shorter than a breast sample constituted mostly of adipose tissue.

By way of example, lung samples 3 mm thick were fixed in 4% formalin and processed by immersion in 40 ml of a solution constituted by 50% paraffin, 20% ethyl alcohol and 30% isoparaffin in an apparatus such as that shown in FIGS. 1 and 2. The samples were subjected to alternating clockwise and counterclockwise rotation, with an initial speed of 250 rpm, increasing by 50 rpm every 15 minutes, up to the maximum speed of 450 rpm, for 60 minutes in total. The procedure allowed to accomplish excellent sample processing, comparable to that of time-consuming and complex traditional processing.

Regarding more complex tissues, breast samples 3 mm thick, for example, were fixed in 4% formalin and processed by immersion in 40 ml of a solution constituted by 50% paraffin, 20% ethyl alcohol, and 30% isoparaffin in an apparatus such as that shown in FIGS. 1 and 2. The samples were subjected to alternating clockwise and counterclockwise rotation, with an initial speed of 250 rpm, increasing by 50 rpm every 30 minutes, up to the maximum speed of 450 rpm, for 120 minutes in total. The procedure allowed to accomplish excellent sample processing, comparable to that of time-consuming and complex traditional processing.

1. A process for processing a biological sample to be subjected to an anatomo-pathological analysis, said process comprising
rotating said biological sample immersed in a processing reagent around its own axis, wherein said axis is straight line passing through the center of mass of the sample in any direction, wherein said rotating step is carried out at a speed from 100 to 1000 rotations per minute.

2. The process according to claim 1, wherein said biological sample is placed inside a container, for housing biological samples.

3. The process according to claim 1, wherein said container containing the biological sample is rotated around its own axis inside a vessel (2) containing the processing reagent, by means (3) suitable for the purpose.

4. The process according to claim 1, wherein said vessels containing the processing reagent are housed in a device provided with means for moving said container around its own axis.

5. The process according to claim 1, wherein said biological sample is processed separately from other biological samples.

6. The process according to claim 1, wherein said rotating step is carried out in any way or direction.

7. The process according to claim 1, wherein said rotating step is carried out at a speed from 200 rotations per minutes to 500 rotations per minute.

8. The process according to claim 7, wherein said speed is modulated during the sample processing.

9. The process according to claim 1, wherein said processing comprises the treatments of fixation and/or dehydration and/or clarification and/or paraffin infiltration, which are carried out separately, sequentially or simultaneously.

10. The process according to claim 9, wherein said treatments are carried out simultaneously, by using a single processing reagent.

11. The process according to claim 10, wherein said single processing reagent is selected from
a mixture of paraffin, at least one alcohol selected from ethyl and isopropyl alcohol and at least one hydrocarbon selected from naphtha and octane;
a mixture of paraffin, at least one alcohol selected from ethyl and isopropyl alcohol and at least one hydrocarbon selected from isoparaffin and limonene;
a mixture of paraffin, ethyl alcohol and isoparaffin.

12. The process according to claim 1, wherein said biological sample is a cytological, histological or autopsical sample to be subjected to an anatomo-pathological analysis.

13. The process according to claim 2, wherein said container is a cassette (1).

14. The process according to claim 6, wherein said direction is longitudinal, transverse, diagonal or sagittal.
